(19) United States
(12) Patent Application Publication
(10) Pub. No.: US 2007/0202047 A1
(43) Pub. Date: Aug. 30, 2007

(54) Polyamine-substituted ligands for use as contrast agents

(76) Inventors: Markus Wolf, Leimen (DE); Ulrike Bauder-Wust, Schriesheim (DE); Uwe Haberkorn, Schwetzingen (DE); Michael Eisenhut, Heidelberg (DE); Walter Mier, Bensheim (DE)

Correspondence Address: Connolly Bove Lodge & Hutz LLP, 1007 North Orange Street, P.O. Box 2207, Wilmington, DE, 19899, US

(21) Appl. No.: 11/649,503

(22) Filed: Jan. 4, 2007

Related U.S. Application Data
(60) Provisional application No. 60/756,352, filed on Jan. 5, 2006.

(51) Int. Cl. A61K 49/10; C07F 5/00; C07D 257/02

(52) U.S. Cl. 424/9.363; 534/15; 540/474

(57) ABSTRACT

The present invention relates to a polyamine-substituted ligand for the preparation of a contrast agent derived from a chelating molecule selected from the group consisting of 1,4,7,10-tetraazacyclododecane-1,4,7,10-tetraacetic acid (DOTA) and diethylentriamine-pentaacetic acid (DTPA), wherein at least one of the carboxylic groups of the chelating molecule is reacted with an amine of formula HNR¹R² to form an amide bond, wherein R¹, R² are independently selected from the group consisting of H; (CH₂)ₙ—NR³R⁴; and R⁵; R³, R⁴ are independently selected from the group consisting of H; (CH₂)ₘ—NR⁶R⁷; and (CH₂)ₘ₋₁—CH₃; R⁶, R⁷ are independently selected from the group consisting of H; and (CH₂)ₒ₋₁—CH₃; n, m, o are independently 2, 3, or 4; R⁵ is of formula 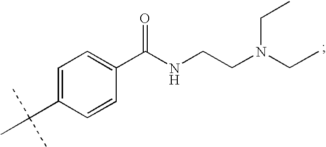
and optionally at least one of the carboxylic groups of the chelating molecule is further reacted with a monoalkylamine having 1 to 18 carbon atoms to form an amide bond; provided that at least one of R¹, R² is other than H. Furthermore, the invention relates to contrast agents for magnetic resonance imaging (MRI) comprising said ligands and in-vivo diagnostic methods based on MRI using said contrast agents.

This application claims benefit of priority from and hereby incorporates by reference in its entirety U.S. Patent Application Ser. No. 60/756,352, filed Jan. 5, 2006.

### FIELD OF THE INVENTION

The present invention relates to polyamine-substituted ligands for the preparation of contrast agents useful in in-vivo diagnostic methods based on magnetic resonance imaging.

### BACKGROUND OF THE INVENTION

Magnetic resonance imaging (MRI) is well known in medical diagnostics. In a strong magnetic field radio-frequency (rf) pulses are used to excite free protons in tissue. After rf excitation relaxation of the magnetization occurs in two different ways. Depending on tissue properties those two effects are described by the time constants, longitudinal (T₁) and transversal (T₂) relaxation time. Usually liquid parts of the tissue are hyperintense in T₂-weighted MR images and hypointense in T₁-weighted MR images. Fatty tissue is hyperintense in both methods. Due to local edema pathologies are often better assessed by T₂-weighted techniques. In contrast, T₂-weighted imaging is higher sensitive to susceptibility artefacts which can occur due to high local blood flow or tissue bleeding. Thus, the morphological assessment is often better on the T₁-weighted images.

To improve the sensitivity and/or specificity of the T₁-weighted imaging technique the application of a contrast agent is advantageous. In MRI normally a paramagnetic metal is used as contrast agent. However, the toxic effects of such a metal have to be avoided. Therefore, such metals are applied in form of a complex with chelating organic ligands. Most commonly used are small chelates of gadolinium, mostly as complex with diethylenetriamine pentaacetic acid (DTPA). They are marked by a fast renal clearance, early extravasation and a low toxicity. This makes them suitable for many clinical implementations such as the detection and delineation of pathologically altered tissue or micro-angiographies of the large circulation.

Another aim is to enhance magnetic resonance imaging (MRI) contrast between normal and diseased tissue or between specific tissue compartments. Therefor a variety of intra- or extravascular paramagnetic contrast agents are available, e.g., the gadolinium(III) chelation complex [Gd(DTPA)(H₂O)]²⁻ (commercial name: Magnevist®; generic name: gadopentetate dimeglumine; DTPA=diethylenetriamine-N,N,N′,N″,N″-pentaacetic acid) or [Gd(DO3A-butrol)(H₂O)] (Gadovist® or gadobutrol; DO3A-butrol=1,4,7-tris(carboxymethyl)-10-(1,2,4-trihydroxy-but-3-yl)-1,4,7,10-tetraazacyclododecane). (See Caravan, P., et al., “Gadolinium (III) chelates as MRI contrast agents: structure, dynamics, and applications,” Chem. Rev. 1999, vol. 99, pp. 2293-352.) By increasing the relaxation rate R₁=1/T₁ of neighboring water protons, such agents enhance the intrinsic contrast between tissues or compartments in T1-weighted MR images in a concentration-dependent manner. Increasing efforts are being made to develop target-specific agents. (See Fulvio, U., et al., “Novel contrast agents for magnetic resonance imaging. Synthesis and characterization of the ligand BOPTA and its Ln(III) complexes (Ln=Gd, La, Lu). X-ray structure of disodium (TPS-9-145337286-C—S)-[4-carboxy-5,8,11-tris(carboxymethyl)-1-phenyl-2-oxa-5,8,11-triazatridecan-13-oato(5-)]gadolinate(2-) in a mixture with its enantiomer,” Inorg. Chem., 1995, vol. 34, pp. 633-42; Ostrowitzki, S., et al., “Comparison of gadopentetate dimeglumine and albumin-(Gd-DTPA)30 for microvessel characterization in an intracranial glioma model,” J. Magn. Reson. Imaging, 1998, vol. 8, pp. 799-806; Schima, W., et al., “MR imaging of the liver with Gd-BOPTA: quantitative analysis of T₁-weighted images at two different doses,” J. Magn. Reson. Imaging, 1999, vol. 10, pp. 80-3; Aime, S., et al., “Targeting cells with MR imaging probes based on paramagnetic Gd(III) chelates,” Curr. Pharm. Biotechnol., 2004, vol. 5, pp. 509-18.) For example, tissue specificity has been achieved with complexes conjugated to monoclonal antibodies. (See Artemov, D., et al., “Molecular magnetic resonance imaging with targeted contrast agents,” J. Cell. Biochem., 2003, vol. 90, pp. 518-24; Shahbazi-Gahrouei, D., et al., “In vitro studies of gadolinium-DTPA conjugated with monoclonal antibodies as cancer-specific magnetic resonance imaging contrast agents,” Australas. Phys. Eng. Sci. Med., 2002, vol. 25, pp. 31-8.) An alternative are folated-dendrimer based contrast agents which bind to the high-affinity folate receptor (hFR) overexpressed in many types of epithelial tumors such as ovarian carcinomas. (See Konda, S. D., et al., “Specific targeting of folate-dendrimer MRI contrast agents to the high affinity folate receptor expressed in ovarian tumor xenografts,” MAGMA, 2001, vol. 12, pp. 104-13.) However, the number of cell-surface antigens or receptors that can be utilized by extracellular, interstitial contrast agents may represent a limitation of this technique.

An alternative strategy is to employ intracellular uptake as a means of “labeling” the cells of interest. 10⁷-10⁸ GD(III) complexes (0.017-0.17 fmol) per cell need to be internalized to achieve a detectable contrast enhancement via T1-weighted MRI. Ideally, the uptake of contrast agent should reflect a specific tissue type or pathophysiologic process of diagnostic significance. However, only a few reports have appeared concerning cellular internalization of gadolinium complexes, which may be attributed to the lack of specific transporters for the currently used contrast agents. (See Konda, S. D., et al., “Specific targeting of folate-dendrimer MRI contrast agents to the high affinity folate receptor expressed in ovarian tumor xenografts,” MAGMA, 2001, vol. 12, pp. 104-13; Allen, M. J., et al., “Cellular delivery of MRI contrast agents,” Chem. Biol., 2004, vol. 11, pp. 301-7; Allen, M. J., et al., “Synthesis and visualization of a membrane-permeable MRI contrast agent,” J. Biol. Inorg. Chem., 2003, vol. 8, pp. 746-50; Bhorade, R., et al., “Macrocyclic chelators with paramagnetic cations are internalized into mammalian cells via a HIV-tat derived membrane translocation peptide,” Bioconjung. Chem., 2000, vol. 11, pp. 301-5.) Intracellular MRI contrast agents employing membrane-penetrating peptides, such as the arginine-rich HIV-tat membrane translocation signal peptide (See Bhorade, R., et al., “Macrocyclic chelators with paramagnetic cations are internalized into mammalian cells via a HIV-tat derived membrane translocation peptide,” Bioconjung. Chem., 2000, vol. 11, pp. 301-5; Prantner, A. M., et al., “Synthesis and characterization of a Gd-DOTA-D-permeation peptide for magnetic resonance relaxation enhancement of intracellular targets,” Mol. Imaging, 2003, vol. 2, pp. 333-41.) or polyarginine oligomers (See Allen, M. J., et al., “Cellular delivery of MRI contrast agents,” Chem. Biol., 2004, vol. 11, pp. 301-7.), lack tissue (e.g. tumor) selectivity. (See Jones, S. W., et al., “Characterization of cell-penetrating peptide-mediated peptide delivery,” Br. J. Pharmacol., 2005, vol. 145, pp. 1093-102.) Stem cells can internalize [Gd(HP-DO3A)(H₂O)] by pinocytosis and have been labeled with this agent in ex vivo incubations. (See Crich, S. G., et al., “Improved route for the visualization of stem cells labeled with a Gd-/Eu-chelate as dual (MRI and fluorescence) agent,” Magn. Reson. Med., 2004, vol. 51, pp. 938-44.) Gd-texaphyrin, a porphyrin-based agent, exhibits tumor cell uptake (See Young, S. W., et al., “Gadolinium(III) texaphyrin: a tumor selective radiation sensitizer that is detectable by MRI,” Proc. Natl. Acad. Sci. USA, 1996, vol. 93, pp. 6610-5. (Erratum in Proc. Natl. Acad. Sci. USA, 1999, vol. 96, pp. 2569.)) with rapid influx and efflux characteristics. (See Heckl, S., et al., “Intracellular visualization of prostate cancer using magnetic resonance imaging,” Cancer Res., 2003, vol. 63, pp. 4766-72.) However, there is a need for an intracellular MRI contrast agent which can serve as a marker for tumor cells in general or for a specific tumor type such as melanoma.

The pharmacophores N-(2-diethylaminoethyl)benzamide and 2-(diethylamino)ethylcarboxamide enhance the intracellular delivery of a series of technetium metal complexes (See Eisenhut, M., et al., “Melanoma uptake of (99 m)Tc complexes containing the N-(2-diethylaminoethyl) benzamide structural element,” J. Med. Chem., 2002, vol. 45, pp. 5802-5; Friebe, M., et al., “99 m Tc]oxotechnetium(V) complexes amine-amide-dithiol chelates with dialkylaminoalkyl substituents as potential diagnostic probes for malignant melanoma,” J. Med. Chem., 2001, vol. 44, pp. 3132-40; Friebe, M., et al., “‘3+1’ mixed-ligand oxotechnetium(V) complexes with affinity for melanoma: synthesis and evaluation in vitro and in vivo,” J. Med. Chem., 2000, vol. 43, pp. 2745-52.) The 2-diethylaminoethyl sidechain was found to be responsible for targeting of benzamide derivatives to melanoma cells (See Eisenhut, M., et al., “Radioiodinated N-(2-diethylaminoethyl)benzamide derivatives with high melanoma uptake: structure-affinity relationships, metabolic fate, and intracellular localization,” J. Med. Chem., 2000, vol.43, pp. 3913-22; Wolf, M., et al., “Alkylating benzamides with melanoma cytotoxicity,” Melanoma Res., 2004, vol. 14, pp. 353-60; Michelot, J. M., et al., “Synthesis and evaluation of new iodine-125 radiopharmaceuticals as potential tracers for malignant melanoma,” J. Nucl. Med., 2001, vol. 32, pp. 1573-80; Michelot, J. M., et al., “Phase II scintigraphic clinical trial of malignant melanoma and metastases with iodine-123-N-(2-diethylaminoethyl 4-iodobenzamide),” J. Nucl. Med., 1993, vol. 34, pp. 1260-6.) High melanin affinity was also found for spermidine-substituted benzamides (See Moreau, M. F., et al., “Synthesis, in vitro binding and biodistribution in B16 melanoma-bearing mice of new iodine-125 spermidine benzamide derivatives,” Nucl. Med. Biol., 2005, vol. 32, pp. 377-84.) or the polyamines themselves. (See Tjalve, H., et al., “Affinity of putrescine, spermidine and spermine for pigmented tissues,” Biochem. Biophys. Res. Commun., 1982, vol. 109, pp. 1116-22.) It has been suggested that the radioiodinated benzamides used for melanoma scintigraphy enter tumor cells not only by passive diffusion but also by active transport via polyamine carriers. (See Seiler, N., et al., “Polyamine transport in mammalian cells: An update,” Int. J. Biochem. Cell. Biol., 1996, vol. 28, pp. 843-61.) Biogenic polyamines (putrescine, spermidine, spermine) are internalized by receptor-mediated active transport processes which can result in the accumulation of millimolar quantities and intra-to-extracellular ratios of polyamines on the order of 1000. (See Porter, C. W., et al., “Aliphatic chain length specificity of the polyamine transport system in ascites L1210 leukemia cells,” Cancer Res., 1984, vol. 44, pp. 126-28; Seiler, N., “Thirty years of polyamine-related approaches to cancer therapy: Retrospect and prospect, Part 2—Structural analogues and derivatives,” Curr. Drug Targets, 2003, vol. 4, pp. 565-85.) Furthermore, when cell proliferation is stimulated, polyamine uptake increases relative to that in nonproliferating tissue. (See Pohjanpelto, P., “Putrescine transport is greatly increased in human fibroblasts initiated to proliferate,” J. Cell. Biol., 1976, vol. 68, pp. 512-20.)

### BRIEF SUMMARY OF THE INVENTION

The inventors of the present invention have found that basic amine substituents such as the known melanoma-seeking pharmacophores or polyamines like 4-amino-N-(2-diethylaminoethyl)benzamide (procainamide) and 2-(diethylamino)ethylamine as well as the bacterial polyamine bis(2-aminoethyl)amine (See Dalla Via, L., “Membrane binding and transport of N-aminoethyl-1,2-diamino ethane (dien) and N-aminopropyl-1,3-diamino propane (propen) by rat liver mitochondria and their effects on membrane permeability transition,” Mol. Membr. Biol., 2004, vol. 21, pp. 109-18.) and the mammalian polyamine N¹-(3-aminopropyl)butane-1,4-diamine (spermidine) are able to facilitate intracellular uptake and retention of 1,4,7,10-tetraazacyclododecane-1,4,7,10-tetraacetic acid (DOTA) and DTPA complexes into tumor cells and elicit melanoma-targeting behavior. Cellular uptake of the synthesized complexes was quantified for human hepatocytes and melanocytes, murine melanoma (B16) and Morris hepatoma (MH3924A) cells in culture. Furthermore, biodistribution and imaging studies were performed with the latter cell line as solid tumors in rats. The polyamine transport system has broad substrate tolerance (See Cullis, P. M., “Probing the mechanism of transport and compartmentalization of polyamines in mammalian cells,” Chem. Biol., 1999, vol. 6, pp. 717-29.) and spermidine conjugates bearing large substituents on the secondary amino group have been found to be good transporter substrates. (See Seiler, N., et al., “Polyamine transport in mammalian cells: An update,” Int. J. Biochem. Cell. Biol., 1996, vol. 28, pp. 843-61; Holley, J., et al., “Uptake and cytotoxicity of novel nitroimidazole-polyamine conjugates in Ehrlich ascites tumor cells,” Biochem. Pharmacol., 1992, vol. 43, pp. 763-69.)

Thus, an object of the present invention is a polyamine-substituted ligand for the preparation of a contrast agent derived from a chelating molecule selected from the group consisting of 1,4,7,10-tetraazacyclododecane-1,4,7,10-tetraacetic acid (DOTA) and diethylentriamine-pentaacetic acid (DTPA), wherein at least one of the carboxylic groups of the chelating molecule is reacted with an amine of formula HNR¹R² to form an amide bond, wherein

R¹, R² are independently selected from the group consisting of H; (CH₂)ₙ—NR³R⁴; and R⁵;

R³, R⁴ are independently selected from the group consisting of H; (CH₂)ₘ—NR⁶R⁷; and (CH₂)ₘ₋₁—CH₃;

R⁶, R⁷ are independently selected from the group consisting of H; and (CH₂)ₒ₋₁—CH₃;

n, m, o are independently 2, 3, or 4;

R⁵ is of formula 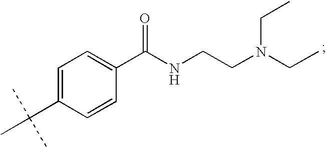
and optionally at least one of the carboxylic groups of the chelating molecule is further reacted with a monoalkylamine having 1 to 18 carbon atoms to form an amide bond; provided that at least one of R¹, R² is other than H.

Another aspect of the present invention is a contrast agent for magnetic resonance imaging (MRI) comprising
(a) a contrast enhancing metal; and
(b) a ligand according to the present invention coordinately bound to the metal.

Yet another aspect of the present invention is an in-vivo diagnostic method based on magnetic resonance imaging (MRI) using a contrast agent according to the present application.

### BRIEF DESCRIPTION OF THE DRAWINGS

The invention is now described in more detail.

In the drawings, FIG. 1 shows the chemical structures of the DTPA-derived ligands 1.1-6.1. The substituents are highlighted with bold face, and the expected net charges of the complexes Gd-1.1. to Gd-6.1. are listed.

FIG. 2 shows the intracellular uptake of the gadolinium complexes Gd-1.1., Gd-2.1. and Gd-4.1. (A) as well as Gd-3.1., Gd-5.1., and Gd-6.1. (B) into cultured B16 melanoma or MH3924A Morris hepatoma cells after 24-h incubation with concentrations in the range 1-10 μM. The uptake of Gd-4.1. into MH3924A cells or the uptake of Magnevist® into both cell lines was below the detection limit of ICP-MS (0.00002 fmol/cell for samples containing 3×10⁶ cells). Approximate intracellular concentrations in μM can be obtained by multiplying the plotted values in fmol/cell by 57. Intracellular uptake of Gd-5.1. in the range 2.5-10 μM and Gd-6.1. in the range 1-10 μM into human hepatocytes (C) and of 10 μM Gd-6.1. into human melanocytes (C) after 24-h incubation.

FIG. 3 shows the Gd-5.1. uptake into MH3924A cells after 1 h incubations at 4° C. and 37° C. (A). Binding inhibition assay: Uptake of 1 μM Gd-5.1. or Gd-6.1. into MH3924A after 24 h incubations at 37° C. in the presence of 1, 10, 25, 50 and 100 μM of the polyamine uptake inhibitor benzyl viologen (B). Subcellular distribution of 100 μM Gd-5.1. in MH3924A after 24 h incubation at 37° C. (C).

FIG. 4 shows the transaxial MR images (2.35 T) which were obtained with T2 weighting (spin echo with TR/TE=2000/32 ms) or T1 weighting (gradient echo with TR/TE=212/5 ms, 60° flip angle) from four anesthesized ACI rats, each bearing a subcutaneous MH3924A tumor in the right thigh. The images from each individual animal (vertical column) were obtained at 1 h or 24 h post intravenous injection of 0.1 mmol/kg of the indicated contrast agent. Regional T₁-weighted contrast enhancement in the partially necrotic tumors is observed at 24 h only with the polyamine-substituted agents Gd-3.1., Gd-5.1. or Gd-6.1. Magnevist® gave results similar to those of Gadovist®.

FIG. 5 shows transverse histological sections (vertical length 8.5 mm) from the central region of a representative MH3924A tumor with staining for cell proliferation (PCNA: darker peripheral regions), vascularization (actin: dark spots in the tumor capsule and periphery and one large transverse vessel) and viable vs. necrotic cells (hematoxylin-eosin (HE): dense, dark regions vs. lighter regions with gaps). The sections are oriented to roughly match the orientation of the tumors in FIG. 4 with the inner face of the tumor at the left.

FIG. 6 shows chemical structures of the DOTA-derived ligands 1.2.-4.2. and the further DTPA-derived ligand 5.2. and the acronyms used for the corresponding Gd complexes.

FIG. 7 shows the intracellular uptake of the gadolinium complex Gd-2.2. into cultured B16 (mouse melanoma), MH3924A (Morris rat hepatoma), Hela (human cervix carcinoma cells), MCF-7 (human breast cancer), A498 (human kidney carcinoma) and mouse fibroblasts (3T3 NIH) after 1-h incubation with concentrations in the range 10-100 μM (A). The uptake of Gadovist® was below the detection limit of ICP-MS (0.01 fmol/cell for samples containing 3×10⁶ cells) (A). Uptake of Gd-3.2. and Gd-4.2. into cultured B16 and MH3924A cells after 1-h incubation with concentrations in the range 10-100 μM (B). Intracellular uptake of the gadolinium complexes Gd-1.2. and Gd-5.2. into cultured B16 (mouse melanoma), MH3924A (Morris rat hepatoma) after 24 h incubation at 1, 2.5, 5 and 10 μM (C).

FIG. 8 shows the biodistribution of Gd-2.2. and Gadovist® in male BALB/c nu/nu mice carrying B16 melanoma (n=1 animal per timepoint). Animals were injected intravenously with Gd-2.2 or Gadovist®, and μg Gd per gram of tissue was measured in perfused tumor and perfused control tissues 1 h post Gd complex injection. The data is given as nmol Gd/g tissue scaled on the dose of 100 μmol of the corresponding Gd complex.

### DETAILED DESCRIPTION OF PREFERRED EMBODIMENTS

As mentioned above, an object of the present invention is a polyamine-substituted ligand for the preparation of a contrast agent derived from a chelating molecule selected from the group consisting of 1,4,7,10-tetraazacyclododecane-1,4,7,10-tetraacetic acid (DOTA) and diethylentriamine-pentaacetic acid (DTPA), wherein at least one of the carboxylic groups of the chelating molecule is reacted with an amine of formula HNR¹R² to form an amide bond, wherein

R¹, R² are independently selected from the group consisting of H; (CH₂)ₙ—NR³R⁴; and R⁵;

R³, R⁴ are independently selected from the group consisting of H; (CH₂)ₘ—NR⁶R⁷; and (CH₂)ₘ₋₁—CH₃;

R⁶, R⁷ are independently selected from the group consisting of H; and (CH₂)ₒ₋₁—CH₃;

n, m, o are independently 2, 3, or 4;

R⁵ is of formula 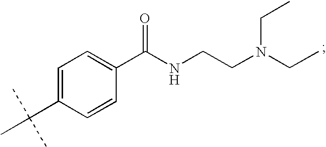
and optionally at least one of the carboxylic groups of the chelating molecule is further reacted with a monoalkylamine having 1 to 18 carbon atoms to form an amide bond; provided that at least one of R¹, R² is other than H.

Thus, the polyamine-substituted ligand is an amide derivative of DOTA or DTPA, wherein one of the carboxylic groups of DOTA or DTPA is reacted with an amine of formula HNR¹R². The chelating molecules DOTA and DTPA are capable to chelate a metal which is necessary in a contrast agent for the magnetic resonance imaging.

Therefore, one aspect of the present invention is a polyamine-substituted ligand, wherein the chelating molecule is DOTA.

Yet another preferred embodiment of the present invention is a polyamine-substituted ligand, wherein the chelating molecule is DTPA.

At least one of the carboxylic groups of DOTA or DTPA is reacted with an amine of formula HNR¹R².

Amide forming reactions are well known in the art. The reaction may be carried out by using well known activation- and/or protection techniques. the one or more carboxylic groups to be reacted may be activated by conversion of the respective carboxylic group in an anhydride functionality or the like. The remaining carboxylic groups may be un-activated or blocked by a suitable protective group. On the other hand the amine may be protected to ensure the desired degree of amidation. Suitable protective groups for amines and carboxylic groups are well known, especially in field of peptide synthesis.

In a preferred embodiment, one or two of the carboxylic groups is reacted with an amine of formula HNR¹R².

In case the chelating molecule is DOTA, it is even more preferred that one of the carboxylic groups of the chelating molecule is reacted with HNR¹R².

In case the chelating molecule is DTPA, it is even more preferred that two of the carboxylic groups of the chelating molecule is reacted with HNR¹R².

The amine used to form an amide with DOTA or DTPA is a formula HNR¹R².

In a preferred embodiment, R¹ is H or (CH₂)ₙ—NH₂ and n 2, 3, or 4.

In a further preferred embodiment, R² is R⁵; (CH₂)ₙ—NH₂; CH₂—CH₂—N(CH₂CH₃)₂; or (CH₂)ₘNH(CH₂)ₒNH₂ and n, m, o are independently 2, 3 or 4.

In a most preferred embodiment, the amine HNR¹R² is selected from the group consisting of

H₂N—R⁵;

H₂N—CH₂—CH₂—N(CH₂CH₃)₂;

HN(CH₂CH₂NH₂)₂;

HN((CH₂CH₂CH₂NH₂)(CH₂CH₂CH₂CH₂NH₂));

HN((CH₂CH₂CH₂NH₂)(CH₂CH₂CH₂CH₂NHCH₂CH₂CH₂NH₂)); and

HN((CH₂CH₂CH₂CH₂NH₂)(CH₂CH₂CH₂NHCH₂CH₂CH₂NH₂)).

It is also possible that optionally at least one of the carboxylic groups of the chelating molecule is further reacted with a monoalkylamine having 1 to 18 carbon atoms to form an amide bound. This additional amide formation may increase the uptake of a contrast agent comprising a ligand of the invention. In a preferred embodiment, such a monoalkylamine is monononylamine.

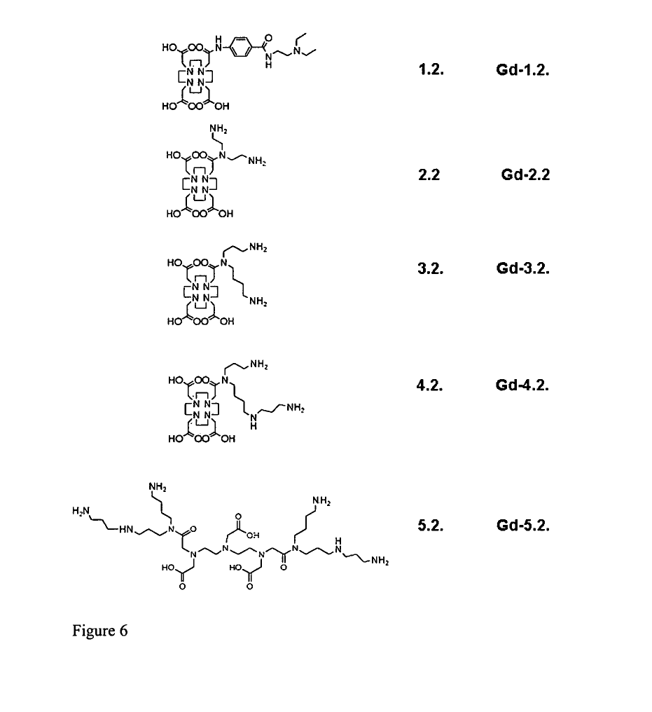

In yet another preferred embodiment the ligands of the invention are those of FIG. 1 and FIG. 6.

Yet another aspect of the present invention is a contrast agent for magnetic resonance imaging (MRI) comprising
(a) a contrast enhancing metal; and
(b) a ligand according to the present invention accordingly bound to the metal.
   Such a contrast agent can be used as described herein. The way of making such a contrast agent is well known in the art.

In a preferred embodiment, the contrast enhancing metal for the contrast agent is gadolinium.

Yet another aspect of the present invention is an in-vivo diagnostic method based on magnetic resonance imaging (MRI) using a contrast agent comprising
(a) a contrast enhancing metal; and
(b) a ligand according to the present invention coordinately bound to the metal.
   In-vivo diagnostic methods applicable for the method of thee present invention are well known in the art. Preferably, the contrast enhancing metal is gadolinium.

The present invention is described in more detail by the following examples which do not limit the scope of the present invention.

### EXAMPLES

### Example 1

Ligand synthesis. All chemicals were purchased from Sigma-Aldrich (Taufkirchen, Germany). The ligands 1.1.-6.1. shown in FIG. 1 were synthesized as briefly described below. Spectroscopic analysis for structure confirmation was performed by electrospray mass spectrometry (ESI-MS, Finnigan TSQ 7000; Thermo Electron Corp, Bremen, Germany) and 250 MHz NMR spectroscopy (AC-250; Bruker BioSpin GmbH, Rheinstetten, Germany). In general, ligand purifications were accomplished by HPLC with Lichrosorb 60 RP Select B columns (250×4 mm, 5 μm, for analytic runs; and 250×10 mm, 10 μm, for preparative runs; Merck KGaA, Darmstadt, Germany) with an eluent flow rate of 3.7 mL/min and ultraviolet detection at 206 nm (SPD-10A VP; Shimadzu, Duisburg, Germany). The eluent comprised 0.1% trifluoroacetic acid (TFA) in water (solvent A) and 0.1% TFA in acetonitrile (solvent B) with a linear gradient of 0% to 100% B in A applied over 30 min. The purity of the products was confirmed by analytical HPLC.

Ligand 1.1 A mixture of DTPA-dianhydride (357 mg, 1 mmol) and procainamide (544 mg, 2 mmol) was stirred in anhydrous DMF for 24 h. Solvent evaporation under reduced pressure gave an oily residue which was purified by preparative HPLC to give 109 mg of 1.1 (yield: 13.2%). The purity was checked by HPLC with two different eluent gradients.

Ligand 2.1. A mixture of DTPA-dianhydride (357 mg, 1 mmol) and 2-(diethylamino)ethylamine (232 mg, 2 mmol) was stirred in anhydrous DMF for 24 h. Solvent evaporation under reduced pressure gave an oily residue which was purified by preparative TLC on silica, using CH₃OH/Et₃N (95/5) as the eluent and ninhydrin as detection reagent, yielding 78 mg of 2.1. (yield: 13.2%).

Ligand 3.1. The primary amino groups of bis(2-aminoethyl)amine (2.5 g, 8.25 mmol) were Boc protected (Boc=t-butyloxycarbonyl) using a method of Rannard and Davis (33) to give 1.58 g of N¹,N³-bis(t-butyloxycarbonyl)-bis(2-aminoethyl)amine (yield: 63%). A mixture of this Boc-protected amine (303 mg, 1 mmol) and 2-(diethylamino)ethylamine (116 mg, 1 mmol) was reacted with DTPA-dianhydride (714 mg, 2 mmol) in anhydrous DMF for 24 h. Purification was accomplished by preparative HPLC. The product-containing fractions were lyophilized to give 37 mg of Boc₂-protected 3.1. as a white powder (yield: 4.8%). Deprotection was performed at ambient temperature for 24 h with a mixture of trifluoroacetic acid, water and triisopropylsilane (90/9/1). Precipitation from methanolic solution with diethylether resulted in 25 mg of 3.1. (yield: 4.3%).

Ligand 4.1. 2-(Diethylamino)ethylamine (116 mg, 1 mmol) and nonylamine (143 mg, 1 mmol) were reacted with DTPA-dianhydride (2 mmol) as above. Workup was performed as described for ligand 2.1. to give 54 mg of 4.1. (yield: 8.8%).

Ligand 5.1. Two equivalents of N¹,N³-bis(Boc)-bis(2-aminoethyl)amine (606 mg, 2 mmol) were reacted with DTPA-dianhydride (1 mmol) as above. Purification of the Boc₄-protected product was accomplished by preparative HPLC. Deprotection and workup was performed as described for ligand 3.1. to give 104 mg of 5.1. (yield: 18.4%).

Ligand 6.1. The primary amino groups of spermidine were selectively protected according to Rannard and Davis (33) to give N¹,N³-bis(Boc)-spermidine, two equivalents of which (700 mg, 2 mmol) were reacted with DTPA-dianhydride (357 mg, 1 mmol) as above. The Boc₄-protected product was purified by preparative HPLC. Deprotection was performed as for ligand 3.1. to give 85 mg of 6.1. (yield: 13%).

Gadolinium complexes. The complexes Gd-1.1 to Gd-6.1. were formed at 90° C. in aqueous solution using gadolinium(III)acetate and equimolar amounts of the corresponding ligands 1.1 to 6.1. Complex Gd-1.1 was purified by preparative reverse-phase HPLC, and purity was judged by the appearance of one single HPLC peak with two different solvent gradients. Complexes Gd-2.1. to Gd-6.1. were obtained from the reaction mixture by solvent evaporation and subsequent crystallization from MeOH and diethylether. Purity was judged by measuring the gadolinium content with ion-coupled plasma (ICP) mass spectrometry (Finnigan Element 2 high-resolution ICP-MS; Thermo Electron Corp., Bremen, Germany). Structure confirmation was obtained by high-resolution ESI-MS with data reported here as measured and (calculated) m/z for the [M+H]⁺ isotopomer with Gd-158.
Gd-1.1.: C₄₀H₅₈GdN₉O₁₀ m/z=983.3684 (983.3620);
Gd-2.1.: C₂₆H₄₈GdN₇O₈ m/z=745.2954 (745.2878);
Gd-3.1.: C₂₄H₄₅GdN₈O₈ m/z=732.3604 (732.2674);
Gd-4.1.: C₂₉H₅₃GdN₆O₈ m/z=772.3307 (772.3239);
Gd-5.1.: C₂₂H₄₂GdN₉O₈ m/z=719.2446 (719.2470);
Gd-6.1.: C₂₈H₅₄GdN₉O₈ m/z=803.3226 (803.3409).

Analytical data for synthesized ligands. Electrospray mass spectrometry (ESI-MS) and NMR spectroscopy were performed for ligands and precursors, as listed below. NMR chemical shifts δ are in ppm relative to TMS; coupling constants J are in Hz.

Ligand 1.1 C₄₀H₆₉N₉O₁₀. ESI-MS: m/z=828.5 for [M+H]⁺ (calc. M=827.45). ¹H-NMR (250 MHz; DMSO-d6/D₂O): δ=1.300 (t, J=7.25, 12H); 3.249 (q, J=7.3, 12 H); 3.341 (t, J=6.3, 4 H); 3.600 (t, J=9.8, 8 H); 3.664 (s, 2 H); 3.714 (s, 4 H); 3.885 (s, 4 H); 7.676 (d, J=9.0, 2 H); 7.732 (d, J=9.0, 2 H); 7.792 (d, J=9.0, 2 H) 7.853 (d, J=9.0, 2 H). ¹³C-NMR (62.9 MHz; DMSO-d6): δ=11.66; 37.28; 46.68; 51.40; 52.07; 59.88; 60.10; 62.81; 69.70; 118.57; 127.58; 128.69; 141.80; 165.70; 170.70; 174.66.

Ligand 2.1. C₂₆H₅₁N₇O₈. ESI-MS: m/z=590.4 for [M+H]⁺ (calc. M=589.38). ¹H-NMR (250 MHz; DMSO-d6/D₂O): δ=1.370 (t, J=7.3, 12 H); 3.144 (t, J=4.9, 4 H); 3.202 (t, J=7.4, 4 H); 3.309 (q, J=4.1, 8 H), 3.367 (q, J=3.7, q); 3.446 (s, 4 H); 3.684 (s, 4 H); 3.846 (s, 2 H). ¹³C-NMR (62.9 MHz; DMSO-d6): δ=7.21; 40.50; 46.24; 48.40; 52.12; 62.90; 171.15; 174.13.

N¹,N³-bis(t-butyloxycarbonyl)-bis(2-aminoethyl)amine. C₁₄H₂₉N₃O₄. ESI-MS: m/z=304.2 for [M+H]⁺ (calc. M=303.22). ¹H-NMR (250 MHz, CDCl₃): δ=1.448 (s, 18 H); 2.726 (q, J=5.5, 4 H); 3.198 (q, J=6.0, 4 H); 5.067 (s, broad, 2 H).

Ligand 3.1. C₂₄H₄₈N₈O₈. ESI-MS: m/z=577.4 for [M+H]⁺ (calc. M=576.36). ¹H-NMR (250 MHz; DMSO-d6/D₂O): δ=1.136 (t, J=7.2, 6 H); 2.853 (q, J=7.2, 4 H); 2.868 (q, J=6.4, 4 H); 2.931 (q, J=5.9, 2 H); 3.018 (t, J=4.3, 8 H); 3.304 (s, 2 H); 3.239 (t, J=4.6, 6 H); 3.623 (s, 8 H). ¹³C-NMR (62.9 MHz; DMSO-d6/D₂O): δ=13.93; 37.28; 39.49; 40.30; 48.69; 50.43; 50.59; 52.98; 56.81; 57.56; 58.24; 171.12; 171.48; 173.59; 174.17; 174.75.

Ligand 4.1. C₂₉H₅₆N₆O₈. ESI-MS: m/z=617.3 for [M+H]⁺ (calc. M=616.42). ¹H-NMR (250 MHz; DMSO-d6/D₂O): δ=0.897 (t, J=6.9, 3 H); 1.241 (t, J=7.4, 6 H); 1.300 (m, J=7.3, 12 H); 1.630 (m, J=7.3, 2 H); 2.914 (t, J=7.8, 8 H); 3.052 (t, J=7.8, 2 H); 3.281 (q, J=7.4, 4 H); 3.331 (s, 2 H); 3.350 (q, J=6.9, 4 H); 3.719 (s, 8 H). ¹³C-NMR (62.9 MHz; DMSO-d6/D₂O): 9.00; 13.93; 22.08; 25.91; 28.58; 28.81; 28.97; 31.27; 38.59; 45.96; 46.27; 50.43; 50.59; 50.82; 50.99; 57.88; 171.30; 173.64; 173.98; 174.24; 174.43.

Boc₄-protected ligand 5.1. C₄₂H₇₇N₉O₁₆. ESI-MS: m/z=964.7 for [M+H]⁺ (calc. M=963.55). ¹H-NMR (250 MHz; DMSO-d6/D₂O/CD₃OD): δ=1.427 (s, 36 H); 3.124 (m, J=5.9, 12 H); 3.353 (t, J=6.0, 12 H); 3.421 (s, 2 H); 3.710 (s, 8 H).

Ligand 5.1. C₂₂H₄₅N₉O₈. ESI-MS: m/z=564.4 for [M+H]⁺ (calc. M=563.34). ¹H-NMR (250 MHz; DMSO-d6/D₂O): δ=2.892 (t, J=11.5, 4 H); 3.026 (t, J=9.6, 8 H); 3.129 (t, J=4.6, 4 H); 3.257 (s, 10 H); 3.520 (t, J=10.1, 8 H). ¹³C-NMR (62.9 MHz; DMSO-d6/D₂O): δ=36.58; 46.40; 48.10; 51.25; 55.00; 58.06; 59.38; 60.19; 173.21; 174.87; 178.53.

N¹,N³-bis(Boc)-spermidine. C₁₇H₃₅N₃O₄. ESI-MS: m/z=346.2 for [M+H]⁺ (calc. M=345.26). ¹H-NMR (250 MHz; CDCl₃): δ=1.438 (s, 18 H); 1.517 (q, J=2.8, 4 H); 1.648 (q, J=6.4, 2 H); 2.655 (q, J=6.4, 4 H); 3.174 (q, J=6.4, 4 H); 5.148 (s, broad, 1 H); 5.420 (s, broad, 1 H).

Boc₄-protected ligand 6.1. C₄₈H₈₉N₉O₁₆. ESI-MS: m/z=1048.8 for [M+H]⁺ (calc. M=1047.64). ¹H-NMR (250 MHz; CD₃OD): δ=1.380 (s, 38 H); 1.762 (m, J=4.5, 12 H); 2.931 (q, J=6.3, 8 H); 3.255 (m, J=5.5, 16 H); 3.584 (s, 6 H); 3.718 (s, 10 H); 6.527 (s, broad, 4 H).

Ligand 6.1. C₂₈H₅₇N₉O₈. ESI-MS: m/z=648.5 for [M+H]⁺ (calc. M=647.43). ¹H-NMR (250 MHz; DMSO-d6/D₂O): δ=1.423 (q, J=3.4, 8 H); 1.636 (q, J=7.7, 4 H); 2.618 (t, J=3.0, 8 H); 2.689 (q, J=5.1, 8 H); 2.836 (t, J=8.5, 8 H); 3.155 (s, 10 H). ¹³C-NMR (62.9 MHz; DMSO-d6/D₂O): δ=25.37; 26.21; 27.61; 38.81; 39.06; 39.40; 39.74; 48.62; 52.16; 52.32; 57.47; 59.46; 176.39; 177.39.

Gadolinium complexes. The content of Gd of the synthesized complexes was determined by elemental analyses.
Gd-2.1.: C₂₆H₄₈GdN₇O₈, Gd content 20.89% (21.14%).
Gd-3.1.: : C₂₄H₄₅GdN₈O₈, Gd content 22.10% (21.51%).
Gd-4.1.: C₂₉H₅₃GdN₆O₈, Gd content 21.2% (20.40%).
Gd-5.1.: C₂₂H₄₂GdN₉O₈, Gd content 22.3% (21.90%).
Gd-6.1.: C₂₈H₅₄GdN₉O₈, Gd content 19.3% (19.61%).

Relaxivity Measurements. A phosphate-buffered saline (PBS, pH 7.4) reference solution and solutions of the Gd complexes Gd-3.1. (60 mM), Gd-5.1. (60 mM), and Gd-6.1. (30 mM) in PBS were placed in glass capillaries (1.2 mm I.D.). Each capillary was then inserted into a standard 5-mm NMR tube containing D₂O (99.96% D) for ¹H relaxation rate measurements at 250 MHz (5.4 T, Bruker AC-250 spectrometer) and 37° C. using the inversion-recovery method. The relaxation rates R₁ were determined by a three-parameter nonlinear least-squares fit of the intensity data to the equation M(τ)=M₀[1−2q exp(−R₁τ)] with 8-11 τ values. The transverse relaxation rate R₂ was estimated from the H₂O signal linewidth Δν at half height (R₂=πΔν). The standard error of estimate for R₁ was <0.7% for all samples. The relaxivities were estimated as r₁ [s⁻¹ mM⁻¹]=[R₁(Gd)−R₁(0)]/[Gd], where [Gd] is the concentration of Gd complex in the sample with relaxation rate R₁(Gd), and R₁(0) is the rate for the reference sample of PBS. Analogous calculations were made for r₂ from R₂.

For the ¹H relaxation rate measurements at 250 MHz (5.4 T, Bruker AC-250 spectrometer) and 37° C., sample temperature was regulated to better than ±0.1° C. using the variable temperature unit of the spectrometer. The solutions studied were placed in capillaries to reduce the sample volume and the resulting intensity of the H₂O signal, thus avoiding the influences of radiation damping on the relaxation behavior. The D₂O solution added to the 5-mm NMR tube provided a field/frequency lock signal and residual HDO as a reference signal. An inversion-recovery pulse sequence (RD-180°-τ-90°-acquisition) was executed with a composite 180° pulse of constant phase, four-step phase cycling of the 90° pulse (7.3 μs) for four transients, and 8-11 τ values. For each sample the list of τ values was chosen after determining the approximate τₙᵤₗₗ for the “null” point of the recovery curve by trial measurements, and the delay RD was set to at least 10τₙᵤₗₗ. The resulting spectra were phase and baseline corrected manually, and the H₂O signal intensity was determined by the peak picking algorithm of the NMR software.

The relaxation rates R₁ were determined by a three-parameter nonlinear least-squares fit of the intensity data to the equation M(τ)=M₀[1−2q exp(−R₁τ)], where q was an additional fitting parameter used to account for the quality of spin inversion. The theoretical value of q is 1.0, and the fitted values were in the range 0.92-0.95. The transverse relaxation rate R₂ was estimated from the H₂O signal linewidth Δν at half height (R₂=πΔν), and the chemical shift Δδ(Gd) of the H₂O signal from the capillary relative to the HDO signal from the external D₂O was determined as an additional measure of the paramagnetism of the Gd complex. The standard error of estimate for R₁was <0.7% for all samples. The relaxivities were estimated as r₁ [s^{31 1} mM⁻¹]=[R₁(Gd)−R₁(0)]/[Gd], where [Gd] is the concentration of Gd complex in the sample with relaxation rate R₁(Gd), and R₁(0) is the rate for the reference sample of PBS. Analogous calculations were made for r₂ from R₂ and for Δδ(Gd).

At 37° C. the fitted relaxation results in s⁻¹ were R₁(0)=0.236±0.005 and R₁(Gd)=469±2 for Gd-3.1. (60 mM), 137.9±0.2 for Gd-5.1. (60 mM), and 194.5±0.9 for Gd-6.1. (30 mM). For the same samples the linewidths gave R₂(0)=1.6 and R₂(Gd)=638 for Gd-3.1., 182 for Gd-5.1., and 86 for Gd-6.1. The molar chemical shifts Δδ(Gd) in ppm mM-⁻¹ were 0.125 for Gd-3.1., 0.045 for Gd-5.1., and 0.161 for Gd-6.1.

Cell culture. B16 melanoma cells (mouse) and MH3924A Morris hepatoma cells (rat) were obtained from the German Cancer Research Center (DKFZ) tumor bank and grown at 37° C. as stock cultures in RPMI 1640 supplemented with 10% fetal calf serum and 1% glutamine as standard medium under a 5% CO₂ atmosphere (all components: Pan Biotech GmbH, Aidenbach, Germany). For contrast agent uptake studies 10⁶ cells were inoculated into 25 cm² culture flasks (BD Biosciences, Bedford, USA) and grown for 24 h in 10 mL of the standard culture medium. Cells were then incubated for an additional 24 h (1 h for temperature dependence studies) with fresh medium containing 0, 1, 2.5, 5 or 10 μM of the selected gadolinium complex (three replicate flasks for each incubation). Afterwards the medium was removed, cells were trypsinated, washed twice, resuspended in culture medium and counted (typical yields: 3.5-6 million cells). For comparison 2.5×10⁵ human melanocytes (Promo Cell, Heidelberg, Germany) were incubated for 24 h with melanocyte growth medium (Promo Cell, Heidelberg, Germany) containing 0, 1, 2.5, 5 or 10 μM of the selected gadolinium complex (two replicate flasks for each incubation). The work up procedure was as described above typically yielding 2.5×10⁵ cells. Cultured human hepatocytes (Cytonet GmbH, Weinheim, Germany) embedded in a collagen matrix in a 12 well microtiter plate, with 6×10⁵ viable cells per well were incubated for 24 h in hepatocyte growth medium (Cytonet GmbH, Weinheim, Germany) containing the same concentration series of selected gadolinium complexes. Afterwards the medium was removed, cells were separated using collagenase (Roche Diagnostics, Mannheim, Germany). After digestion the cells were washed twice with PBS, resuspended in culture medium and counted (typical yields: 6×10⁵ cells per tube). The intracellular gadolinium content was determined by ICP-MS as described below.

Polyamine transport inhibition. 10⁶ MH3924A cells were inoculated into 25 cm² culture flasks (BD Biosciences, Bedford, USA) and grown for 24 h in 10 mL of the standard culture medium. Cells were then incubated for an additional 24 h with fresh medium containing 1 μM of Gd-5.1. or Gd-6.1. and 0, 1, 10, 25, 50 or 100 μM of the polyamine uptake inhibitor benzyl viologen (34, 35). Afterwards the medium was removed, cells were trypsinated, washed twice, resuspended in culture medium and counted (typical yields: 3.5-6 million cells). The gadolinium content of the cells was determined by ICP-MS as described below.

Subcellular distribution. 5×10⁶ MH3924A cells were grown in 25 cm² culture flasks (BD Biosciences, Bedford, USA) for 24 h in 10 mL of the standard culture medium. Cells were then incubated for an additional 24 h with 100 μM Gd-5.1. Afterwards the medium was removed, cells were trypsinated. Then the organelles were isolated with the Subcellular Proteome Extraction Kit ProteoExtract® (Merck, Darmstadt, Germany) according to the instructions of the manufacturer. Four fractions were obtained: Cytosol, membrane organelles (mainly mitochondria), nucleus and cytoskeleton (with associated endosomes and lysosomes). The gadolinium content for each fraction was determined by ICP-MS as described below.

ICP-MS. Samples containing the above mentioned number of harvested cells were digested with 50% concentrated HNO₃ (Superselect; Merck, Darmstadt, Germany) under microwave heating with a Mars 5 apparatus (CEM GmbH, Kamp-Lintfort, Germany). To each sample 100 μL of an aqueous solution of rhodium chloride (1 μg/mL) were added so that Rh-103 could be used as an internal quantification standard. Gd-160 measurements were performed with the ICP-MS instrument mentioned above. The gadolinium concentrations were determined using standard curves created prior to cell analyses. For each uptake incubation condition triplicate cell samples were analyzed, and Gd content for each sample was recorded as the mean of two MS determinations.

Cytotoxicity studies. B16 or MH3924A cells were cultured for 24 h in a 96-well microtiter plate with the medium and standard conditions described above. The culture medium was then exchanged with medium containing the chosen contrast agent at concentrations of 0, 1, 2.5, 5, 10, 25, 75 or 100 μM, and the cells were incubated for an additional 48 h. The fraction of surviving cells was measured by the non-radioactive MTS viability assay (Cell Titer 96® Aqᵤₑₒᵤₛ; Promega, Madison, Wis.) according to the instructions of the manufacturer. The LDH-induced formazan dye formation was determined by optical absorption at 490 nm with a microplate reader (model 3350-UV, Biorad Laboratories GmbH, Munich, Germany). Cell survival was expressed as % of controls for n=4 determinations.

Serum albumin binding. Solutions containing 20 μM Gd-3.1., Gd-5.1. or Gd-6.1. and 40 g/L human serum albumin (Behring, Bern, Switzerland) were incubated at 37° C. for 30 or 90 min. The solutions were subjected to ultrafiltration (filter cutoff 20 kD; Sartorius, Göttingen, Germany), and the amount of unbound complex was determined in the ultrafiltrate with ICP-MS (n=2).

Animal models. For in vivo tumor studies 2×10⁶ MH3924A tumor cells were injected subcutaneously into the right thigh of male ACI rats (Charles River, Sulzfeld, Germany) weighing 220 to 260 g. All animal experiments were performed in compliance with the German Animal Protection Laws (Permission 35-9185.81/G-7-03, Reg.-Praesidium, Karlsruhe, Germany).

Biodistribution and excretion data. At 14 days after tumor inoculation, when tumors had reached a diameter of ca. 5-6 mm, the biodistribution experiments were initiated. Each animal (210-250 g) was given an intravenous tail-vein injection of 0.22 μmol/kg Magnevist® (Schering, Berlin, Germany) or 0.022 μmol/kg of Gd-5.1. or Gd-6.1. 1 h and 24 hs post-injection (p.i.), the animals were weighed, sacrificed by cervical dislocation and dissected. Organs or tissues were blotted dry and weighed. Samples containing 100-500 mg of the tumor or organs were digested with concentrated HNO₃ (Superselect; Merck, Darmstadt, Germany) and H₂O₂ under microwave heating with a Mars 5 apparatus (CEM GmbH, Kamp-Lintfort, Germany). To each sample 100 μL of an aqueous solution of rhodium chloride (1 μg/mL) were added so that Rh-103 could be used as an internal quantification standard. Gd-160 measurements were performed with the ICP-MS instrument mentioned above. The gadolinium concentrations were determined using standard curves created prior to tissue analyses. The results were expressed as % ID/g tissue. Urine and faeces were gathered in order to measure the excreted amount of Gd(III).

MRI. At 25 days after inoculation, when tumors had reached a diameter of ca. 10-15 mm, the MR imaging experiments were initiated. ACI rats were anesthetized by intravenous injection of ketamin (Ketanest®, 0.1 mg/g body wt.; Parke-Davis, Berlin, Germany). Then a dose of 0.1 mmol/kg Magnevist® or Gadovist® (Schering, Berlin, Germany) or one of the synthesized agents Gd-3.1., Gd-5.1. or Gd-6.1. was injected intravenously into a lateral tail vein. For one experiment a higher dose (0.36 mmol/kg) of Magnevist® was used. Individual rats were examined by MRI at 1 h and 24 h post Gd, in the case of Magnevist® also at ca. 15 min post Gd. Proton imaging studies were performed at 2.35 T (100.3 MHz) with a Biospec 24/40 instrument (Bruker BioSpin MRI, Ettlingen, Germany). An actively shielded gradient coil with an inner diameter of 120 cm was used, and the RF resonator had an inner diameter of 92 mm. Two transaxial multislice imaging protocols were applied with a field of view of 70×70 mm, 128 time-domain data points, 96 phase-encoding steps, and 16 slices of 2 mm thickness. A multi-spin-echo sequence was used for proton-density and T₂-weighted images (repetition time TR=2 s, 12 echoes with echo times TE of 8, 16, 24, . . . 96 ms, number of averages=1), and a spoiled gradient-echo sequence was used for T₁-weighted imaging (TR=212 ms, TE=5 ms, flip angle=60°, number of averages=3). The data were Fourier transformed to give a 128×128 image matrix with 0.55 mm pixel resolution. Analogous T₁-weighted images with coronal slices were acquired with a field of view of 80×80 mm and 3-mm slice thickness.

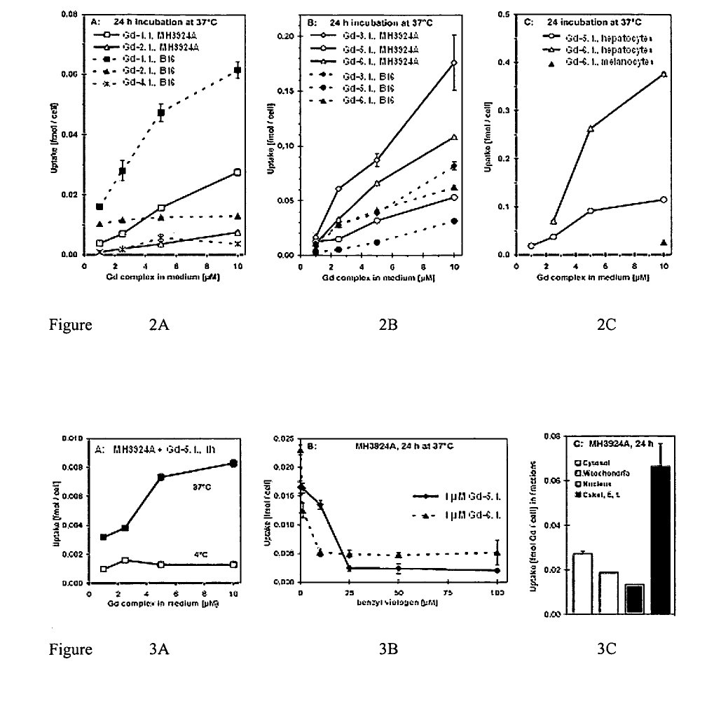

Imaging viewing, ROI analysis and export of TIFF files was performed with the freeware MRIcro from Chris Rorden (http://people.cas.sc.edu/rorden/mricro.html). For FIG. 3 the TIFF files were interpolated to 512×512 pixels in Photoshop® 6.0 (Adobe Systems GmbH, Munich, Germany), and minimal brightness and contrast adjustments were made to give comparable gray scales for all images.

Tissue preparation and histochemistry. Tumor-bearing animals were killed and tumors were dissected, shock-frozen in nitrogen, and stored at −70° C. prior to histochemistry. Cryosectioning (6 μm) was performed with a Microm microtome, and sections were mounted on Superfrost microscope slides (Fisher Scientific, Pittsburgh, Pa.), exposed to acetone (10 min, −20° C.), and dried (30 min). Three types of histochemical staining were performed for: (a) tumor vascularization, using mouse monoclonal antibody anti-alpha-actin; 1:500 (BD Biosciences Pharmingen, San Diego, Calif.); (b) proliferating cells, using mouse antibody against proliferating cell nuclear antigen (PCNA, clone PC10, 1:100; DakoCytomation GmbH, Hamburg, Germany); (c) necrosis, using hematoxylin-eosin. The sections were photographed with an Axioplan2 imaging microscope fitted with the high-resolution digital imaging system AxioCam/AxioVision (Carl Zeiss GmbH, Jena, Germany).

Relaxivity Measurements. Detailed information on the relaxivity of complex solutions and intracellular relaxivity are presented in the Supplementary Data available online.

Statistics. For comparison of means in two treatment groups, p values are given for a two-sided t-test assuming unequal variances.

Results

Syntheses. The symmetric or asymmetric bis(amide) ligands 1.1.-6.1., summarized in FIG. 1, were obtained by straightforward aminolysis reactions of DTPA-dianhydride with one or two of the selected polyamine compounds mentioned above. Boc protection of primary amino groups (33) was applied where necessary. After deprotection the ligands were complexed with Gd^{3+,} and the complexes Gd-1.1 to Gd-6.1. were purified by HPLC or obtained pure as precipitates from the reaction mixture. Structure confirmation was provided by NMR spectroscopy for the ligands (see Supplementary Data online) and by high-resolution ESI-MS for the complexes.

Intracellular Uptake of Gadolinium Complexes. B16 melanoma and MH3924A hepatoma cells as well as human melanocytes and hepatocytes were incubated for 24 h in the presence of the gadolinium complexes shown in FIG. 1, using concentrations in the range of 1-10 μM. Incubations with the unsubstituted [Gd(DTPA)(H₂O)]²⁻complex (Magnevist®) served as controls. Following cell harvest and hydrolysis, inductively coupled plasma mass spectrometry (ICP-MS) with direct detection of gadolinium was used to quantitate complex uptake (triplicate cultures for each incubation).

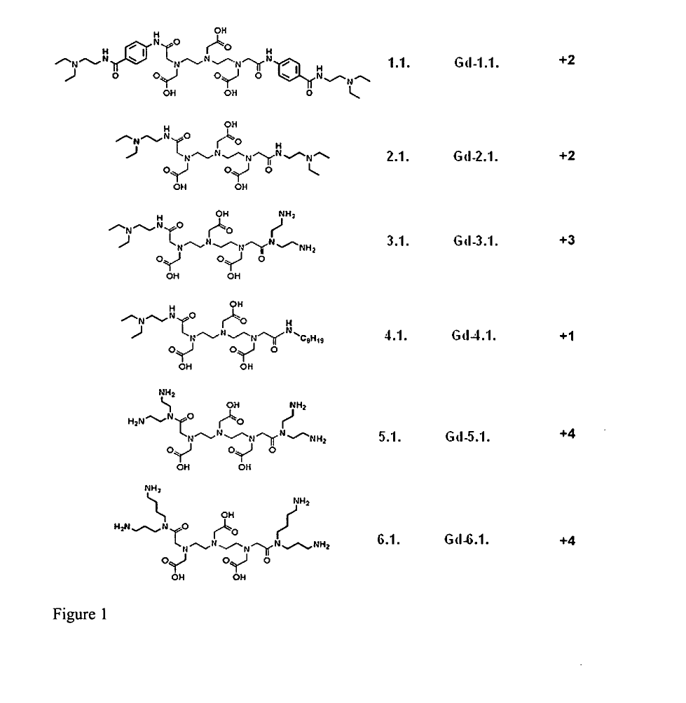

The uptake of [Gd(DTPA)(H₂O)]²⁻proved to be below the ICP-MS detection limit (<0.0002 fmol/cell) for both tumor cell types. The uptake of the complexes Gd-1.1 and Gd-2.1. after 24-h incubation was concentration dependent (FIG. 2A) and reached values in the range 0.062-0.0129 fmol/cell for the 10 μM incubation for B16 melanoma cells. The cellular uptake was about a factor 2.2 higher for Gd-1.1 and a factor 1.72 higher for Gd-2.1. in B16 melanoma vs. the hepatoma cells (FIG. 2A). Replacement of one 2-(diethylamino)ethylamide group (ligand 2.1.) with a bis(2-aminoethyl)amide moiety (ligand 3.1. ) resulted in a 2.87 fold increase in intracellular accumulation of the gadolinium complexes, now with a significant preference for the hepatoma cells. At 10 μM Gd-3.1. uptake was 0.175±0.0013 fmol/cell in MH3924A and 0.082±0.004 fmol/cell in B16 (p<0.001, FIG. 2B). The uptake of Gd-5.1., containing two bis(2-aminoethyl)amide substituents, into MH3924A cells (0.053±0.005 fmol/cell at 10 μM) and B16 cells (0.027±0.002 fmol/cell at 10 μM) was less than that of Gd-3.1. (FIG. 2B). The complex Gd-6.1., which features two spermidine substituents, exhibited uptake of 0.108± into MH3924A. The uptake of Gd-6.1. into B16 melanoma was about a factor of 2 higher versus human melanocytes (FIG. 2C). Finally, the enhancement of the lipophilicity of the complex through the introduction of a nonylamide function (ligand 4.1.) resulted in a neglectable uptake of 0.0036 fmol/cell and was near the detection limit of ICP-MS. The data presented in FIG. 2 represent the actual measured amounts of gadolinium complex detected in 3×10⁶ cells, expressed as fmol/cell. The mean diameter of harvested Morris hepatoma cells was estimated to be 15±1 μm, yielding a mean cell volume of ca. 1.77 pL. Therefore, a gadolinium content of 0.1 fmol/cell corresponds to a concentration of ca. 57 μM, and the uptake achieved for Gd-3.1., Gd-5.1., and Gd-6.1. after 24-h with an incubation concentration of 10 μM (FIG. 2B) corresponds to intracellular concentrations in the range 30 to 100 μM. The intra-to-extracellular concentration ratio (up to a factor 10) provides evidence for an active transport process. The less efficient uptake of Gd-1.1 and Gd-2.1. (FIG. 2A) resulted in calculated intracellular concentrations of 35-7.4 μM with 24-h incubations at 10 μM.

Uptake inhibition experiments with the polyamine uptake inhibitor benzyl viologen (34, 35), the determination of the subcellular distribution and uptake experiments at different temperatures were performed in order to determine the uptake mechanism of the agents. FIG. 3A demonstrates that the uptake of Gd-5.1. into MH3924A cells over 1 h was strongly reduced at 4° C. vs. 37° C. (0.008±0.00041 vs. 0.00064±0.00004 fmol/cell for 10 μM incubations, p<0.001) (FIG. 3A). When cells that had been tested at 4° C. were subsequently incubated at 37° C., uptake of Gd-5.1. was restored to the levels observed with freshly harvested cells. The temperature dependence of passive diffusion alone is proportional to T/η, where η is the solvent viscosity at temperature T. This leads to the prediction that the uptake rate should decrease by at least a factor of 2.5 at 4° C. vs. 37° C. The observed decrease in uptake shown in FIG. 3A is about a factor of 6.5.

In order to demonstrate that these agents are incorporated into cancer cells using the polyamine receptors a binding inhibition assay was performed. The uptake of 1 μM of Gd-5.1. and Gd-6.1. into MH3924A was determined in the presence of increasing amounts of the polyamine uptake inhibitor benzyl viologen (in the range from 1 μM up to 100 μM). At a 25 fold molar excess of benzyl viologen up to 90% of the Gd complex uptake could be inhibited (FIG. 3B). The IC₅₀ values were 16.8 μM for Gd-5.1. and 29.9 μM for Gd-6.1. Furthermore subcellular concentrations of Gd-5.1. in MH3924A cells were found to range between 0.015 and 0.026 fmol/cell in the nucleus and cytoplasma, respectively. The highest concentration of 0.065 fmol/cell was detected in the cytoskeleton fraction (FIG. 3C).

Cellular toxicity. Cellular toxicities of Gd-3.1., Gd-5.1., Gd-6.1. and Magnevist® were tested in an MTS assay with MH3924A cells. Incubations with gadolinium complex at concentrations of 1-100 μM were performed for 48 h, and cell survival (percentage relative to untreated controls) ranged between 90% and 118%. There were no significant differences between different complexes or between complex and control (data not shown).

Serum albumin binding. Incubation of Gd-3.1., Gd-5.1. or Gd-6.1. with human serum albumin for 30 or 90 min at 37° C. (see Methods) resulted in no significant binding of complex to protein, e.g., 93±6% of Gd-3.1. was found to be free. This situation was considered to be favorable for in vivo studies.

Biodistribution and excretion data. ACI rats bearing a subcutaneous MH3924A tumor in the right thigh received an intravenous bolus injection (0.22 μmol/kg) of the commercial extracellular contrast agent Magnevist® or 0.022 μmol of the polyamine-substituted complex Gd-5.1. or Gd-6.1. The Gd content of organs and the tumor was determined 1 h and 24 h after the injection of the contrast agent and is given as % ID/g organ (Table 1). Biodistribution data showed that Gd-5.1. binds preferentially to lung tissue and parts of the excretory system e.g. small intestine and kidneys. There was a significant tumor preference compared with the liver values 1 h post injection. 24 h post injection, Magnevist® was completely cleared from the tumor whereas about 0.020 fmol/cell of polyamine-substituted Gd-DTPA agent Gd-5.1. were still present in the tumor. Similar results were obtained with Gd-6.1. Furthermore urine and faeces were collected and the gadolinium content of the excrements was measured. Using Magnevist® 88% of the gadolinium was found in the urine one hour post injection whereas 98% was obtained in the urine and about 1% in the faeces 24 h after injecting Gd-5.1. and Gd-6.1.

Relaxivity of gadolinium complexes in solution. The longitudinal relaxation rate R₁=1T₁ of water protons in a stock solution (phosphate-buffered saline) containing Gd-3.1., Gd-5.1., or Gd-6.1. was measured at 5.4 T (250 MHz) using conventional inversion-recovery techniques. The transverse relaxation rates were estimated from the measured linewidth Δν of the water signal (R₂=πΔν). After subtraction of R₁and R₂ for reference buffer solutions, the following molar relaxivities r₁ in units of s⁻¹ mm⁻¹ at 37° C. and 250 MHz were calculated: 7.8 for Gd-3.1. , 6.5 for Gd-6.1., 2.3 for Gd-5.1. The ratios r₂/r₁ were ca. 1.3 for all three complexes. For comparison, r₁=ca. 4.0 for the common agents with DTPA or DO3A ligands (1).

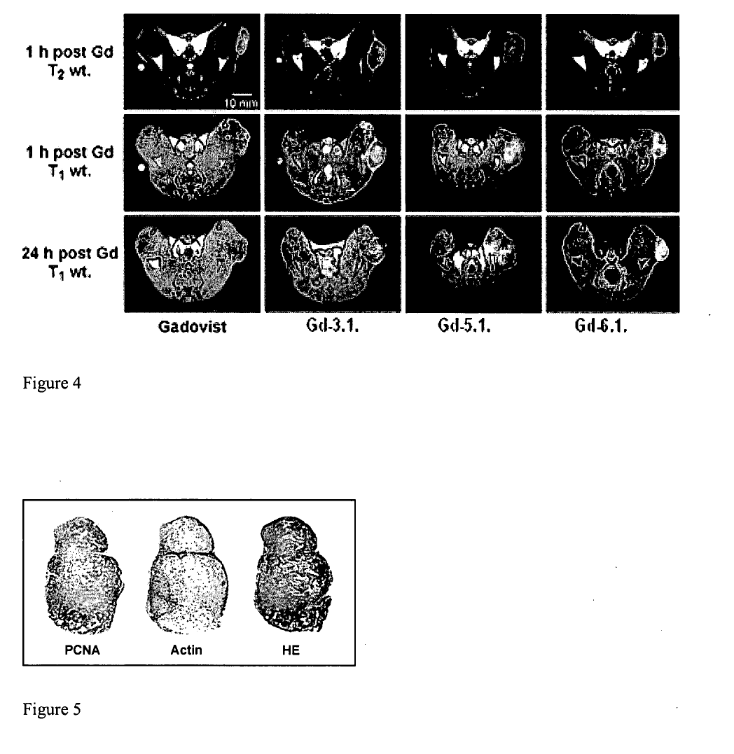

Magnetic Resonance Imaging. A pilot series of MRI experiments with ACI rats bearing a subcutaneous MH3924A tumor in the right thigh was performed at 2.35 T. The anesthetized animals received an intravenous bolus injection (0.1 mmol/kg) of the commercial extracellular contrast agent Magnevist® or Gadovist® or one of the polyamine-substituted complexes Gd-3.1., Gd-5.1., or Gd-6.1. In addition one experiment was performed with a high dose of Magnevist® (0.36 mmol/kg). Multislice transverse and coronal images were obtained 1 h post Gd with either T₁- or T₂-weighting and after 24 h with T₁-weighting. Individual transverse slices covering the central tumor region are compared in FIG. 4.

With T₂ weighting the tumor can be delineated as a hyperintense area, independent of contrast agent, with a contrast ratio of 2.0-2.3 relative to neighboring muscle tissue. With T₁ weighting at 1 h post Gd, there is low contrast for tumor vs. muscle tissue with Gadovist® (max. contrast ratio=1.06; similar results for Magnevist®, not shown) while accumulation of the polyamine complexes can be visualized as regional hyperintensity in the tumor with contrast ratios of 1.22, 1.11 and 1.49 for Gd-3.1., Gd-5.1. and Gd-6.1., respectively. At this time point all contrast agents showed high accumulation in the bladder, as expected for clearance via the kidneys. The images of FIG. 4 also demonstrate that even after 24 h sufficient concentrations of polyamine complex are still present in tumor, resulting in regional hyperintensity with contrast ratios of 1.11, 1.08 and 1.21 for Gd-3.1., Gd-5.1. and Gd-6.1., respectively. With Magnevist® hyperintensity in tumor could be detected at 10-15 min post Gd and also at 1 h when a 3.6-fold higher dose was used (data not shown), but in all cases at 24 h the extracellular agent was cleared from tumor, and no contrast with T₁ weighting was achieved.

At 1 h post Gd the T₂-weighted images shown in FIG. 4 (TE=32 ms) exhibit moderate intensity variations within tumor that show the same pattern in the images at TE=8 ms and are, therefore, mainly due to spin density variations. For the polyamine contrast agents the images at TE=96 ms show hypointense regions (shortest T₂) which coincide with the hyperintense regions (shortest T₁) in the displayed T₁-weighted images. In general, the tumors exhibited heterogeneous uptake of polyamine contrast agent, with large signal intensity variations within and between the individual image slices. For Gd-3.1. or Gd-6.1. in FIG. 3, the tumor periphery and internal regions showed hyperintensity with T₁ weighting at 1 h post Gd.

Histology. Some of the tumors studied by MRI were excised for histological examination. In general, the tumors exhibited a well-define capsule, a peripheral zone of vascularization (alph-actin staining) and proliferating cells (PCNA staining), and one or more central regions of necrosis (hematoxylin-eosin staining). An example is shown in FIG. 5, where the tumor is not identical with one of those in FIG. 4 but the central histological slices are oriented to match the basic orientation in the MR images. The general histology was similar for all tumors examined, with the extent of necrosis increasing with tumor size.

Example 1 shows that the extracellular MR contrast agent gadolinium DTPA (Magnevist®) can be transformed into a membrane-permeable intracellular agent by the introducing two positively charged functional groups such as procainamide or 2-(diethylamino)ethylamine to the DTPA ligand. Thus, the intracellular uptake of Gd-1.1 into B16 or MH3924A tumor cells reached concentrations on the order of 35 μM (ca. 0.062 fmol/cell, FIG. 2A) after 24-h incubations with 10 μM contrast agent. Cellular uptake is boosted, leading to intracellular concentrations in the range of 100 μM, when the polyamine bis(2-aminoethyl)amine or spermidine is employed as DTPA substituent. Of the complexes tested Gd-3.1. showed the highest uptake into MH3924A cells, achieving 0.175 fmol/cell with a 24-h, 10μM incubation. Although the three polyamine-substituted DTPA complexes exhibited somewhat higher affinity for the Morris hepatoma cells, uptake into B16 melanoma cells was also substantial (FIG. 2B) and sufficient for contrast enhancement in MR imaging applications. The goal of developing gadolinium complexes with melanoma affinity by introducing melanin-binding pharmacophores into the sidechains of DTPA was partially realized with Gd-1.1 and Gd-2.1. The higher accumulation of these complexes in B16 and MH3924A cells presumably involves melanosome binding and can be attributed to the 2-(diethylamino)ethylamine terminal substituents in ligand 1.1 Analogous behavior was observed with technetium complexes containing similar structural elements (17-19). With the introduction of one polyamine substituent in Gd-3.1., at the expense of one melanin-binding pharmacophore, a remarkable increase in uptake was achieved for both cell lines, presumably due to increased affinity for the polyamine transporter, while the preference for melanoma cells was lost. The positively charged polyamine substituents employed in this study promote facilitated transport of gadolinium DTPA complexes through the cell membrane, leading to a intra-to-extracellular concentration gradient of up to 10:1. The transport is an energy-dependent process so that uptake should decrease significantly at 4° C., as demonstrated in FIG. 3.

It is known that endosomes and lysosomes are bound to the cytoskeleton (36). This means that Gd(III) is localised in the endosomes and lysosomes. That is the compartment where polyamines are localised as well (31). The subcellular distribution gives further evidence for a polyamine transporter mediated uptake of the agents. Efficient inhibition of Gd-5.1. and Gd-6.1. uptake and the preferential localization of the complexes in the endosomes and lysosomes of MH3924A cells provide further proof for the assumption that these agents are internalized using the polyamine transporters. These finding may be interpreted as an indication that the modified Gd-DTPA complexes are imported via polyamine transporters, which are typically upregulated in highly proliferating or malignant cells such as the MH3924A and B16 lines chosen for this study (26, 29).

The organ distribution study showed that Gd-5.1. and Gd-6.1. accumulated in the lungs and small intestine. This correlates with high levels of biogenic polyamines found in these organs (37). The liver values are elevated and correspond with the high amounts of spermidine found in the liver (37). Despite the high liver value a significant tumor preference was found at least at 1 h post injection. Even after 24 h Gd-5.1. was retained in the organs and tumor, whereas Magnevist® was nearly completely eliminated beyond 1 h post injection. Therefore, the novel Gd-complexes show a comparable organ distribution in regard to the organ concentrations of natural polyamines. Gd-5.1. is almost completely excreted through the kidneys. The bulk amount of the injected complex is, however, collected through this organ as known from Magnevist®.

The polyamine-substituted Gd complexes proved to be nontoxic for cultured MH3924A cells at concentrations up to 100 μM and exhibited favorable relaxivity characteristics. Therefore, these complexes were considered safe and suitable for initial imaging experiments. Thus, assuming the necessary permeability of the tumor vasculature, sufficient uptake of the polyamine-substituted Gd complexes for tumor-specific MRI contrast enhancement at bolus doses of 100 μmol/kg was expected and is confirmed by the results in FIG. 4. The retention of the polyamine contrast agents in tumor even after 24 h has been demonstrated and could not be achieved with conventional extracellular agents such as Magnevist® or Gadovist®.

In summary, example 1 illustrates the potential of utilizing the polyamine transporters of proliferating cells, for active, facilitated uptake of MRI contrast agents in tumors in general.

TABLE 1
Biodistribution of Magnevist ®, Gd-5, and Gd-6 (conc. in
nmol/g) and cumulative excretion for tumor-bearing ACI rats.*

| | Magnevist, | Gd-5, | Gd-5, | Gd-6, | Gd-6, |
|---|---|---|---|---|---|
| Tissue | 1 h | 1 h | 24 h | 1 h | 24 h |
| Tumor (MH3924A) | 3.1 | 5.5 | 0.6 | 7.2 | 0.8 |
| Liver | 3.0 | 4.2 | 2.0 | 6.6 | 5.5 |
| Kidney (+urine) | 40.7 | 120.3 | 40.9 | 141.6 | 62.3 |
| Lung | 4.1 | 12.7 | 24.2 | 20.2 | 5.9 |
| Blood | 4.8 | 7.8 | 0.1 | 4.8 | 0.1 |
| Muscle (femur) | 1.3 | 1.6 | n.a. | n.a. | n.a. |
| Spleen | 1.5 | 4.7 | n.a. | n.a. | n.a. |
| Bone (femur) | 0.8 | 2.2 | 0.9 | 3.3 | 1.0 |
| Bone Marrow (femur) | 1.5 | 0.8 | 1.7 | 0.9 | 2.3 |
| Small Intestine | 1.8 | 18.9 | 0.4 | 4.0 | 0.1 |
| Brain | 0.4 | 0.5 | n.a. | n.a. | n.a. |
| Urine (cumulative) | 25805.3 | 2116.7 | 537.2 | n.a. | 650.9 |
| Faeces | n.a. | n.a. | 16.6 | n.a. | 41.4 |
| Excretion as % of total dose | n.a. | n.a. | 98 | n.a. | 94 |

*Rats were sacrificed 1 h or 24 h post-injection of contrast agent; tissues and biofluid samples were hydrolyzed and analyzed by ICP-MS to give Gd concentration in nmol/g tissue or fluid.
Each column of data represents results for one animal, scaled proportionally to correspond to a constant Gd dose of 100 μmol/kg body wt.; n.a. = sample not available. After 24 h Magnevist was below the ICP-MS detection limit (ca. 0.03 nmol/g) for tumor, liver, or kidney.

### Example 2

Ligand synthesis. All chemicals were purchased from Sigma-Aldrich (Taufkirchen, Germany). The ligands 1.2.-5.2. shown in FIG. 6 were synthesized as briefly described below. Spectroscopic analysis for structure confirmation was performed by electrospray mass spectrometry (ESI-MS, Finnigan TSQ 7000; Thermo Electron Corp, Bremen, Germany) and 250 MHz NMR spectroscopy (AC-250; Bruker BioSpin GmbH, Rheinstetten, Germany). In general, ligand purifications were accomplished by HPLC with Lichrosorb 60 RP Select B columns (250×4 mm, 5 μm, for analytic runs; and 250×10 mm, 10 μm, for preparative runs; Merck KGaA, Darmstadt, Germany) with an eluent flow rate of 3.7 mL/min and ultraviolet detection at 214 nm (SPD-10A VP; Shimadzu, Duisburg, Germany). The eluent comprised 0.1% trifluoroacetic acid (TFA) in water (solvent A) and 0.1% TFA in acetonitrile (solvent B) with a linear gradient of 0% to 100% B in A applied over 30 min. Structure confirmation was given by ¹H-NMR and ESI-MS. The purity of the products was confirmed by analytical HPLC.

1.2.: DOTA-tris(t-butyl ester) (573 mg, 1 mmol) was activated with HATU (380 mg, 1 mmol) in CH₃CN (3 mL) for 5 min and subsequently reacted with a mixture of procainamide (272 mg, 1 mmol) and ethyl-diisopropylamine (340 μL) dissolved in CH₃CN (2 mL). Stirring for 3 h at ambient temperature, solvent evaporation and liquid chromatography on silica using CH₂Cl₂/CH₃OH/Et₃N (70/28/2) as the eluent afforded the DOTA-tris(t-butyl ester)-procainamide conjugate as an oil (340 mg, 43%). ESI-MS: m/z 791.1 [M+H]⁺ (calc. M=790.1). Deprotection of the t-butyl ester groups was performed with a mixture of trifluoroacetic acid, water and triisopropylsilane (90/9/1) for 24 hours at ambient temperature. Crystallization from methanol/ethylacetate/chloroform (1/1/1) yielded DOTA-procainamide 1.2. as a white precipitate (42 mg, 17%). C₂₉H₄₇N₇O₈. ESI-MS: m/z 622.4 for [M+H]⁺ (calc. M=621.7). ¹H-NMR (250 MHz; CDOD₃): δ=1.378 (t, J=7.298 Hz, 6H); 3.349 (m, J=2.92 Hz, 24 H); 3.789(t, J=6,171 Hz, 2 H); 3.919 (q, J=6.156 Hz, 2H); 4.095 (q, J=7.12 Hz, 4H); 7.884 (d, J=0.454 Hz, 2 H); 7.900 (d, J=0.415 Hz, 2H).

2.2.: DOTA (500 mg, 1.14 mmol) was dissolved in 10 mL of water. A solution of bis(2-aminoethyl)amine (120 mg, 1.14 mmol) in 8 mL of acetonitril was added. A solution of N,N′-dicyclohexylcarbodiimide (DCC) (235 mg, 1.14 mmol) in 8 mL pyridine was added dropwise with stirring. The reaction mixture was stirred for an additional 3 hours at ambient temperature. The reaction mixture was evaporated to dryness under reduced pressure. The residue was taken up in 20% acetonitril in water. The resulting suspension was filtered. The filtrate was purified by preparative HPLC. Lyophylisation yielded 48 mg (8.6%). C₂₀H₃₉N₇O₇. ESI-MS: m/z 490.4 for [M+H]⁺ (calc. M=489.29). ¹H-NMR (250 MHz; CDOD₃): δ=2.930 (m, J=5.97 Hz, 16 H); 3.237 (s, 8 H); 3.567 (t, J=5.97 Hz, 4 H); 3.981 (t, J=6.77 Hz, 4 H).

3.2.: DOTA-tris(t-butyl ester) (573 mg, 1 mmol) was activated with HATU (380 mg, 1 mmol) in DMF (3 mL) for 5 min and subsequently reacted with a mixture of spermidine (145 mg, 1 mmol) and ethyl-diisopropylamine (340 μL) dissolved in DMF (2 mL). Stirring for 3 h at ambient temperature and solvent evaporation afforded the DOTA-tris(t-butyl ester)-spermidine conjugate. Deprotection of the t-butyl ester groups was performed with a mixture of trifluoroacetic acid, water and triisopropylsilane (90/9/1) for 24 hours at ambient temperature. Preparative HPLC and lyophylisation yielded DOTA-spermidine 3.2 as a white precipitate (23 mg, 4.3%). C₂₃H₄₅N₇O₇. MALDI-MS: 532.15 for [M+H]⁺ (calc. M=531.31). ¹H-NMR (250 MHz, CDOD₃): δ=1.718 (q, J=3.62 Hz, 4 H); 1.994 (q, J=5.55 Hz, 2 H); 2.904 (m, J=1.7 Hz, 8 H); 2.949 (m, J=4.05 Hz, 16 H); 3.533 (s, broad, 8 H).

4.2.: DOTA-tris(t-butyl ester) (573 mg, 1 mmol) was activated with HATU (380 mg, 1 mmol) in DMF (3 mL) for 5 min and subsequently reacted with a mixture of spermine (202 mg, 1 mmol) and ethyl-diisopropylamine (340 μL) dissolved in DMF (2 mL). Stirring for 3 h at ambient temperature and solvent evaporation and afforded the DOTA-tris(t-butyl ester)-spermine conjugate. Deprotection of the t-butyl ester groups was performed with a mixture of trifluoroacetic acid, water and triisopropylsilane (90/9/1) for 24 hours at ambient temperature. Preparative HPLC and lyophylisation yielded DOTA-spermine 4.2. as a white precipitate (22 mg, 3.7%). C₂₆H₅₂N₈O₇. MALDI-MS: 589.39 for [M+H]⁺ (calc. M=588.74). ¹H-NMR (δ, D₂O): 1.804 (m, J=5.12 Hz, 4 H); 2.093 (m, J=3.88 Hz, 4 H); 3.131 (m, J=3.77 Hz, 16 H), 3.303 (s, broad, 14 H); 3.870 (s, broad, 6 H).

5.2.: The primary amino groups of spermidine were selectively protected using a method of Rannard et al. (38) to obtain Boc₂-spermine. ESI-MS: m/z 403.3 for [M+H⁺]. ¹H-NMR (δ, CDCl₃): 1.648 (s, 18 H); 1.620 (m, 4 H); 1.700 (m, 4 H); 2.053 (s [broad], 2 H); 2.600 (m, 8 H); 3.194 (q, 4 H), 5.262 (s [broad], 2 H).

Two equivalents of Boc₂-spermine were reacted with DTPA-dianhydride (1 mmol). The product was purified by preparative HPLC at 206 nm. ESI-MS: n/z 1162.9 for [M+H⁺]. ¹H-NMR (δ, D₂O, d-DMSO): 0.758 (s, 36 H); 1.083 (m, J=4.66 Hz, 12 H); 2.457 (m, J=3.76 Hz, 16 H); 2.658 (t, J=3.67 Hz, 8 H); 3.031 (s, 10 H).

Deprotection of the Boc groups was performed with a mixture of trifluoroacetic acid, water and triisopropylsilane (90/9/1) for 24 hours at ambient temperature. The crude product was precipitated with diethylether. Crystallization from methanol/isopropanol/chloroform yielded 33 mg (4.33%) of a white powder.

C₃₄H₇₁N₁₁O₈, ESI-MS: m/z 762.6 for [M+H⁺] (calc. M=761.55). ¹H-NMR (δ, D₂O, d-DMSO): 1.776 (m, J=5.44 Hz, 8 H); 1.864 (m, J=3.67 Hz, 8 H); 2.827 (m, J=3.67 Hz, 24 H); 3.027 (m, J=6.88 Hz, 8 H); 3.277 (s, 6H); 3.438 (s, 4H).

Gadolinium complexes. The complexes Gd-1.2. to Gd-5.2. were formed at 90° C. in aqueous solution using gadolinium(III)acetate and equimolar amounts of the corresponding ligands 1.2. to 5.2. Complex Gd-1.2. was purified by preparative reverse-phase HPLC, and purity was judged by the appearance of one single HPLC peak with two different solvent gradients. Free Gd³⁺ was precipitated from the reaction mixture by addition of NaOH. Complexes Gd-2.2. to Gd-5.2. were obtained from the reaction mixture—after removal of uncomplexed Gd³⁺—by solvent evaporation and subsequent crystallization from MeOH and diethylether. Purity was judged by measuring the gadolinium content with ion-coupled plasma (ICP) mass spectrometry (Finnigan Element 2 high-resolution ICP-MS; Thermo Electron Corp., Bremen, Germany). Structure confirmation was obtained by high-resolution ESI-MS with data reported here as measured and (calculated) m/z for the [M+H]⁺ isotopomer with Gd-158.
Gd-1.2.: C₄₀H₅₈GdN₉O₁₀ m/z=777.2574 (777.2571);
Gd-2.2.: C₂₀H₃₆GdN₇O₇ m/z=645.1946 (644.1943);
Gd-3.2.: C₂₄H₄₅GdN₈O₈ m/z=687.2399 (686.2403);
Gd-4.2.: C₂₉H₅₃GdN₆O₈ m/z=744.3021 (743.2982);
Gd-5.2.: C₂₂H₄₂GdN₉O₈ m/z=917.4521 (916.4510).

Purity was judged by the appearance of one single HPLC peak with two different solvent gradients and elemental analysis (Quantification of Gd).

| | | |
|---|---|---|
| Gd-1.2.: | 19.89% | (calc. 20.21%) |
| Gd-2.2.: | 24.67% | (calc. 24.40%) |
| Gd-3.2.: | 23.22% | (calc. 22.90%) |
| Gd-4.2.: | 21.09% | (calc. 21.12%) |
| Gd-5.2.: | 17.10% | (calc. 17.13%). |

Complex stability. The stability constants were determined using a method of Sherry et al. (39).

Cytotoxicity studies. B16 or MH3924A cells were cultured for 24 h in a 96-well microtiter plate with the medium and standard conditions described above. The culture medium was then exchanged with medium containing the chosen contrast agent at concentrations of 0, 1, 2.5, 5, 10, 25, 75 or 100 μM, and the cells were incubated for an additional 48 h. The fraction of surviving cells was measured by the non-radioactive MTS viability assay (Cell Titer 96® Aqueous; Promega, Madison, Wis.) according to the instructions of the manufacturer. The LDH-induced formazan dye formation was determined by optical absorption at 490 nm with a microplate reader (model 3350-UV, Biorad Laboratories GmbH, Munich, Germany). Cell survival was expressed as % of controls for n=4 determinations.

Cell Uptake Studies. A493 (human kidney), B16 melanoma cells (mouse), HeLa (human cervix), MCF-7 (human breats), MH3924A (Morris hepatoma) cells (rat) and 3T3 NIH (mouse fibroblasts) were obtained from the DKFZ tumor cell collection. 3×10⁶ cells of each cell type were grown for 24 hours in culture flasks. Afterwards they were incubated for 1 hour in 10 mL culture media containing 0, 10, 50 and 100 μM of Gd-2.2. Gd-3.2 or Gd-4.2. Afterwards the medium was removed, the cells were trypsinated, washed twice, resuspended in culture medium and counted. The gadolinium uptake was determined in triplicate. For The cells were incubated with 0, 1, 2.5, 5 and 10 μM of the Gd-1.2. and Gd-5.2. for 24 h.

The intracellular retention of Gd-2.2. was determined as follows: 3×10⁶ cells of each cell type were grown for 24 hours in culture flasks. Afterwards they were incubated for 1 hour in 10 mL culture media containing 0, 10, 50 and 100 μM of the respective gadolinium complexes. Afterwards the medium was removed, fresh medium was added and an additional 24 hours was waited until the cells were trypsinated, washed twice, resuspended in culture medium and counted. The gadolinium uptake was determined in triplicate.

ICP-MS: Equal cell counts were taken and digested with HNO₃ under microwave heating (Mars5, CEM). The samples were diluted with water containing Rh-103 as an internal standard. Gd-157 measurements were performed with a high resolution ICP-mass spectrometer (ELEMENT 2, Finnigan MAT). The gadolinium concentrations were determined from standard curves created prior to sample analyses.

HSA Binding: 20 μM solutions of the complexes were incubated at 37° C. for 30 and 90 minutes in a solution of 40 g HSA/L (Behring, Bern; Switzerland). The amount of unbound complex was determined in ultrafiltrates with ICP-MS. Ultrafilters had a cutoff of 20 kD (Sartorius, Göttingen, Germany).

Animal model. For in vivo tumor studies 3×10⁶ B16 tumor cells were injected subcutaneously into the right thigh of male BALB/c nu/nu mice (Charles River, Sulzfeld, Germany) weighing 18 to 21 g. All animal experiments were performed in compliance with the German Animal Protection Laws (Permission 35-9185.81/G-7-03, Reg.-Praesidium, Karlsruhe, Germany).

Biodistribution data. At 10 days after tumor inoculation, when tumors had reached a diameter of ca. 7-8 mm, the biodistribution experiments were initiated. Each animal (18-21 g) was given an intravenous tail-vein injection of 975 μmol/kg Gadovist® (Schering, Berlin, Germany) or 1 μmol/kg of Gd-2.2. 1 h post-injection (p.i.), the animals were weighed, sacrificed by cervical dislocation, perfused and dissected. Organs or tissues were blotted dry and weighed. Samples containing the tumor or organs were digested with concentrated HNO₃ (Superselect; Merck, Darmstadt, Germany) and H₂O₂ under microwave heating with a Mars 5 apparatus (CEM GmbH, Kamp-Lintfort, Germany). To each sample 100 μL of an aqueous solution of rhodium chloride (1 μg/mL) were added so that Rh-103 could be used as an internal quantification standard. Gd-160 measurements were performed with the ICP-MS instrument mentioned above. The gadolinium concentrations were determined using standard curves created prior to tissue analyses. The results are expressed as nmol Gd/g tissue scaled on the dose of 100 μmol of the corresponding Gd complex.

Statistics. For comparison of means in two treatment groups, p values are given for a two-sided t-test assuming unequal variances.

Results

Ligand synthesis. The DOTA ligands were obtained by HATU mediated condensation of DOTA-(tris tert. Butylester) with the corresponding amine. Deprotection with TFA yielded the polyamine substituted ligands. The symmetric ligand 5.2. was obtained by straightforward aminolysis reactions of DTPA-dianhydride with spermine. Boc protection of primary amino groups (33) was applied where necessary. After deprotection the ligands were complexed with Gd³⁺.

Complex stability. The complex stability constants for Gd-2.2. and Gd-5.2. were determined. The complex stability constant for Gd-2.2. was about 3.45×10²¹ and the one of Gd-5.2. was 4.22×10¹⁵. The DOTA ligands are more stable than the DTPA ligands.

Cellular toxicity. Cellular toxicities of the new complexes and Gadovist® were tested in an MTS assay with MH3924A cells. Incubations with gadolinium complex at concentrations of 1-100 μM were performed for 48 h, and cell survival (percentage relative to untreated controls) ranged between 90% and 118%. There were no significant differences between different complexes or between complex and control (data not shown).

Serum albumin binding. Incubation of the complexes with human serum albumin for 30 or 90 min at 37° C. (see Methods) resulted in no significant binding of complex to protein, e.g., 92±5% of Gd-2.2. was found to be free. This situation was considered to be favorable for in vivo studies.

Intracellular Uptake of Gadolinium Complexes. B16 melanoma and MH3924A hepatoma cells were incubated for 24 h in the presence of the gadolinium complexes Gd-1.2. and Gd-5.2. shown in FIG. 6. In the case of Gd-2.2., Gd-3.2 or Gd-4.2. B16 melanoma, MH3924A hepatoma cells were incubated for 1 h in the presence of the gadolinium complexes using concentrations in the range of 10-100 μM. Additionally the cellular uptake of Gd-2.2. into A498, HeLa, MCF-7 and 3T3 NIH mouse fibroblasts was determined. Incubations with the unsubstituted [Gd(DOTA)(H₂O)]⁻ complex served as control. Following cell harvest and hydrolysis, inductively coupled plasma mass spectrometry (ICP-MS) with direct detection of gadolinium was used to quantitate complex uptake (triplicate cultures for each incubation).

The uptake of [Gd(DOTA)(H₂O)]⁻ proved to be below the ICP-MS detection limit (<0.0002 fmol/cell) for both tumor cell types. The uptake of the complex Gd-2.2. after 1 h incubation was concentration dependent (FIG. 7A) and reached values in the range 0.222 fmol/cell (MH3924A) following 1 h incubation with 100 μM complex. Suffient intracellular amounts of Gd-2.2. for MR imaging purposes were achieved in B16 melanoma cells (0.057 fmol/cell) and human kidney carcinoma A498 (0.023 fmol/cell). The uptake of Gd-2.2. into MCF-7 (0.0086 fmol/cell) and HeLa (0.0078 fmol/cell) cells would be insufficient for MRI imaging.

In order to get information about the ability of the cells to release the complex we performed a cell uptake experiment in which after a 1 h incubation of MH3924A with Gd-2.2. the medium was refreshed with medium containing no complex. After an additional 24 h the cells were harvested. The intracellular gadolinium content was the same as for the 1 h incubation indicating that the complexes are not externalized by the cells. Uptake of Gd-4.2. into MH3924A and B16 melanoma cells was comparable with the uptake of Gd-2.2., but somewhat lower. Uptake of Gd-4.2. was lower than uptake of Gd-3.2. and Gd-2.2., but still sufficient for MRI imaging after 1 h incubation with 100 μM of the complex. The uptake of Gd-1.2. was sufficient for MRI imaging purposes but was the lowest of all tested complexes. The uptake of Gd-5.2. into both cell lines reached values of 0.3 fmol/cell for MH3924A and 0.03 fmol/cell for B16 melanoma cells.

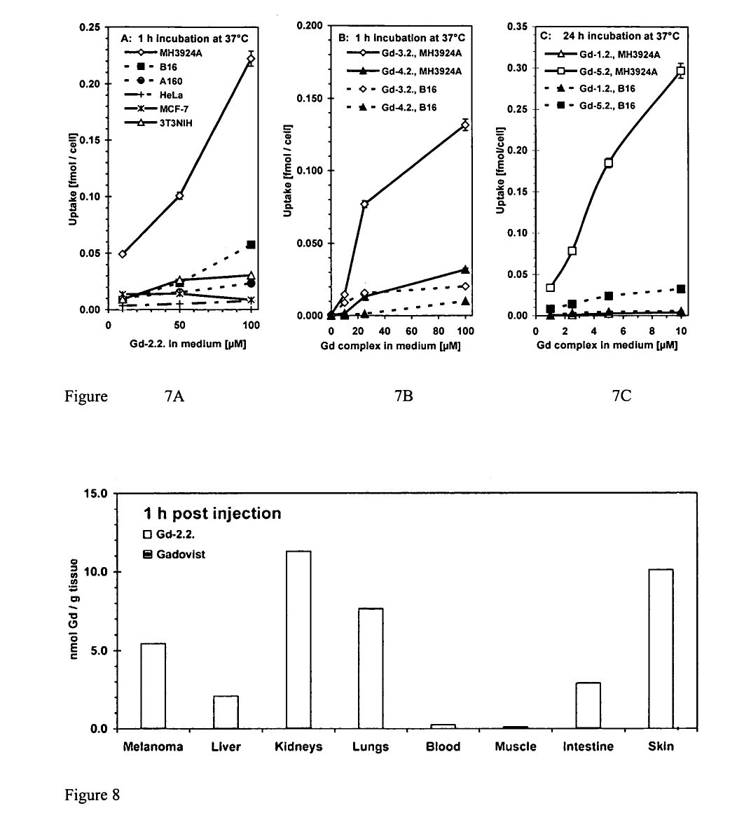

Biodistribution studies. Male Balb C nu/nu mice with a subcutaneous B16 melanoma in the right thigh received an intravenous bolus injection (975 μmol/kg) of the commercial extracellular contrast agent Gadovist® or 1 μmol of the polyamine-substituted complex Gd-2.2. The Gd content of organs and the tumor was determined 1 h after the injection of the contrast agent and perfusion of the organs (FIG. 8). Biodistribution data showed that Gd-2.2. binds preferentially to kidneys and skin tissue, but shows high tumor uptake as well. Gadovist® was completely cleared from the tumor 1 h post injection.

1. A polyamine-substituted ligand for the preparation of a contrast agent derived from a chelating molecule selected from the group consisting of 1,4,7,10-tetraazacyclododecane-1,4,7,10-tetraacetic acid (DOTA) and diethylentriamine-pentaacetic acid (DTPA), wherein at least one of the carboxylic groups of the chelating molecule is reacted with an amine of formula HNR¹R² to form an amide bond, wherein
R¹, R² are independently selected from the group consisting of H; (CH₂)ₙ—NR³R⁴; and R⁵;
R³, R⁴ are independently selected from the group consisting of H; (CH₂)ₘ—NR⁶R⁷; and (CH₂)ₘ₋₁—CH₃;
R⁶, R⁷ are independently selected from the group consisting of H; and (CH₂)ₒ₋₁—CH₃;
n, m, o are independently 2, 3, or 4;
R⁵ is of formula 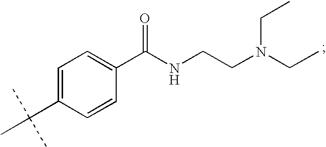
and optionally at least one of the carboxylic groups of the chelating molecule is further reacted with a monoalkylamine having 1 to 18 carbon atoms to form an amide bond;
provided that at least one of R¹, R² is other than H.

2. The ligand of claim 1, wherein the chelating molecule is DOTA.

3. The ligand of claim 1, wherein the chelating molecule is DTPA.

4. The ligand of claim 1, wherein one or two of the carboxylic groups of the chelating molecule is reacted with HNR¹R².

5. The ligand of claim 2, wherein one of the carboxylic groups of the chelating molecule is reacted with HNR¹R².

6. The ligand of claim 3, wherein two of the carboxylic groups of the chelating molecule is reacted with HNR¹R².

7. The ligand of claim 1, wherein R¹is H or (CH₂)ₙ—NH₂ and n 2, 3, or 4.

8. The ligand of claim 1, wherein R² is R⁵; (CH₂)ₙ—NH₂; CH₂—CH₂—N(CH₂CH₃)₂; or (CH₂)ₘNH(CH₂)ₒNH₂ and n, m, o are independently 2, 3 or 4.

9. The ligand of claim 1, wherein HNR¹R² is selected from the group consisting of
H₂N—R⁵;
H₂N—CH₂—CH₂—N(CH₂CH₃)₂;
HN(CH₂CH₂NH₂)₂;
HN((CH₂CH₂CH₂NH₂)(CH₂CH₂CH₂CH₂NH₂));
HN((CH₂CH₂CH₂NH₂)(CH₂CH₂CH₂CH₂NHCH₂CH₂CH₂NH₂)); and
HN((CH₂CH₂CH₂CH₂NH₂)(CH₂CH₂CH₂NHCH₂CH₂CH₂NH₂)).

10. The ligand of claim 1, wherein the monoalkylamine is monononylamine.

11. A contrast agent for magnetic resonance imaging (MRI) comprising
(a) a contrast enhancing metal; and
(b) a ligand according to claim 1 coordinately bound to the metal.

12. The agent of claim 11, wherein the contrast enhancing metal is gadolinium.

13. An in-vivo diagnostic method based on magnetic resonance imaging (MRI) using a contrast agent comprising
(a) a contrast enhancing metal; and
(b) a ligand according to claim 1 coordinately bound to the metal.

14. The method of claim 13, wherein the contrast enhancing metal is gadolinium.
